(19) United States
(12) Patent Application Publication
(10) Pub. No.: US 2004/0236378 A1
(43) Pub. Date: Nov. 25, 2004

(54) Pacemaker for atrial sensing, atrial stimulation and termination of atrial tachycardias and auricular fibrillation, and a method of controlling a cardiac pacemaker

(75) Inventor: Wolfgang Hartung, Hannover (DE)

Correspondence Address: HAHN LOESER & PARKS, LLP, TWIN OAKS ESTATE, 1225 W. MARKET STREET, AKRON, OH, 44313, US

(73) Assignee: Biotronik Mess-und Therapiegeraete GmbH & Co. Ingenieurbuero Berlin

(21) Appl. No.: 10/674,279

(22) Filed: Sep. 29, 2003

(30) Foreign Application Priority Data
Sep. 30, 2002 (DE) 102 45 852.9

(51) Int. Cl. A61N 1/362

(52) U.S. Cl. 607/9

(57) ABSTRACT

In a cardiac pacemaker arrangement comprising an electrode arranged floatingly in the atrium, a circuit for perceiving atrial signals, and a circuit for stimulating the atrial myocardium by means of the electrode, the invention proposes that in addition there is provided a wall-located electrode, and stimulation is effected by means of the wall-located electrode if the circuit, upon perceiving atrial signals, does not detect high-frequency irregularities—such as auricular fibrillation or atrial tachycardias—as on the basis of inadmissibly high signal frequencies—, and stimulation is effected by means of the floating electrode if the circuit, upon perceiving atrial signals, detects irregularities of that kind. This permits early detection and termination of atrial tachycardias and auricular fibrillation.

### TECHNICAL FIELD

The invention concerns a cardiac pacemaker arrangement as set forth in the classifying portion of claim 1 and a method of controlling a cardiac pacemaker as set forth in the classifying portion of claim 6.

### BACKGROUND OF THE INVENTION

Auricular fibrillation which occurs paroxysmally—that is to say in the nature of sudden attacks—nowadays represents a clinical challenge. According to the respective literature source involved it is assumed that up to 10% of all patients over 60 years suffer from auricular fibrillation. Hitherto auricular fibrillation is deemed to be incurable. There is a series of therapeutic approaches—from drug therapy through cardiac pacemaker therapy and defibrillator therapy to various ablation procedures—all of which however still fail to give satisfactory results.

In the field of cardiac pacemaker therapy there are various stimulation algorithms or stimulation configurations which are intended to prevent the occurrence of auricular fibrillation. Various algorithms have been developed for the termination of auricular fibrillation, but hitherto they have not proven themselves.

The object of the present invention is to improve a cardiac pacemaker arrangement of the general kind set forth, in such a way that it permits early detection and termination of atrial tachycardias and auricular fibrillation, and to provide a method suited thereto of controlling a cardiac pacemaker.

### BRIEF SUMMARY OF THE INVENTION

That object is attained by an arrangement having the features of claim 1 and a method having the features of claim 8.

The invention proposes in other words that three essential aspects are joined together: firstly signal perception by means of floating electrodes, secondly the combination of floating and wall-located electrodes, and thirdly a circuit which in dependence on the perceived signals controls the stimulation by way of different electrodes.

In this respect the pacemaker arrangement can operate in two different modes:

in mode 1 (sensing-pacing mode) the pacemaker arrangement perceives the atrial signals by way of floating and/or wall-located electrodes and permits per se known and proven fit and healthy stimulation by way of the wall-located electrode or electrodes, and

in mode 2 (pacing-termination mode) the cardiac pacemaker arrangement permits particularly large-area stimulation of the atrial myocardium, which can be suitable for the termination of auricular fibrillation and atrial tachycardia. That stimulation is effected in the form of atrial floating stimulation by means of conventional stimulation configurations or by means of newer floating configurations which are known by the names OLBI or BIMOS. Stimulation can be effected exclusively by way of the floating electrode or electrodes or also by way of a combination of floating electrode or electrodes, with a wall-located electrode or electrodes respectively.

### BRIEF DESCRIPTION OF SEVERAL VIEWS OF THE DRAWINGS

Those two modes are discussed in greater detail hereinafter with reference to the drawings in which:

FIG. 1 shows a comparison of atrial signal perception by means of floating ring electrodes and by means of wall-located electrodes,

FIG. 2 shows a comparison of the atrial intrinsic sensing commencement for different electrode arrangements,

FIG. 3 shows illustrations of different electrodes and—beneath same—the illustrations associated with those arrangements of intracardial derivations,

FIG. 4 shows a diagrammatic view of an embodiment of a pacemaker arrangement as proposed,

FIG. 5 shows a view of the atrial simultaneous activation surface in stimulation in accordance with the proposal, and

FIG. 6 shows a measuring protocol for the termination of auricular fibrillation.

### DETAILED DESCRIPTION OF THE INVENTION

Mode 1: Sensing-Pacing Mode

The perception of atrial signals in pacemaker therapy which involves the right atrium is usually effected either by way of wall-located electrodes (conventional AAI or DDD pacemaker principle) or by way of floating atrial electrodes (conventional VDD pacemaker principle). The stability and dependability of atrial perception have been described for both principles in a large number of studies. Intra-individual comparison of the two concepts has hitherto not been effected.

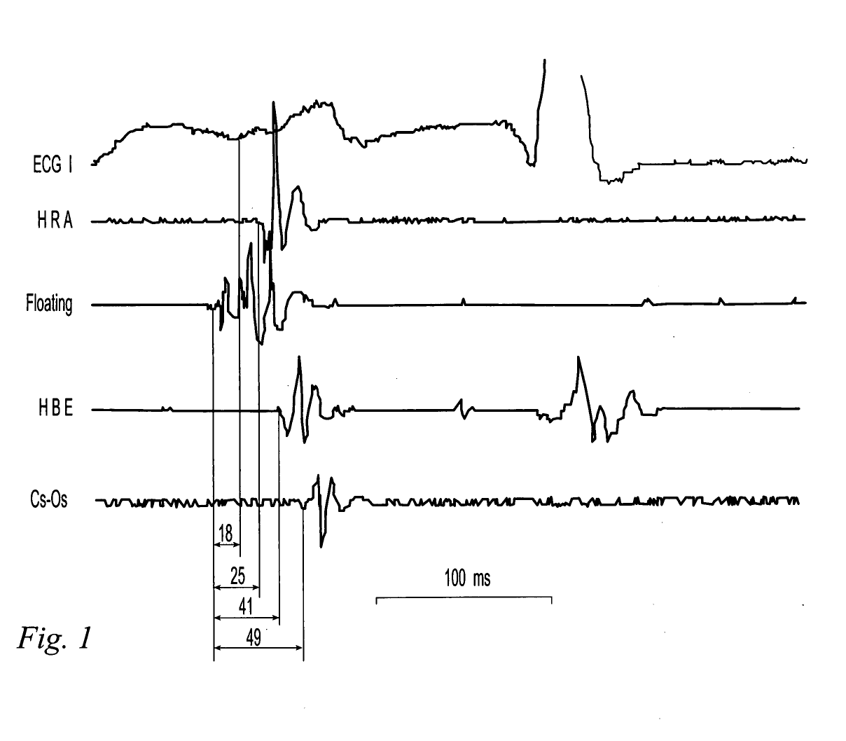

In the applicant's own animal-experiment investigations it was possible for the first time to show the advantage of atrial signal perception by way of floating ring electrodes in comparison with wall-located electrodes. FIG. 1 shows an example of simultaneous registration of bipolar sensing of the intrinsic activation times (in ms) both using atrial electrodes with wall contact in the high lateral night atrium (“HRA”), at the ostium of the coronary sinus (“Cs—Os”) and at the His's bundle (“HABE”) and also by way of floating electrodes of a VDD-electrode in the central right atrium (“Floating”), in which respect it can be seen from FIG. 1 that atrial signals are perceived by way of the floating electrodes earlier than by way of the wall-located electrodes, irrespective of the placement in the atrium:

It was found in that animal-experimental study that atrial signals are perceived by way of floating ring electrodes as follows:

22±4 ms (p<0.05) earlier than the commencement of the P-wave in the surfaces—ECG der. 1,

and 22±5 ms (p<0.05) earlier than the earliest perception by way of the wall-located electrodes in the high right atrium (HRA: typical wall-located electrode positioning in conventional pacemaker therapy),

and 36±13 ms earlier in comparison with the His's bundle position (HBE) (p<0.05),

and finally 43±8 ms earlier (p<0.05) with respect to the electrode positioning at the coronary sinus ostium (Cs—Os or lower right atrium=URA).

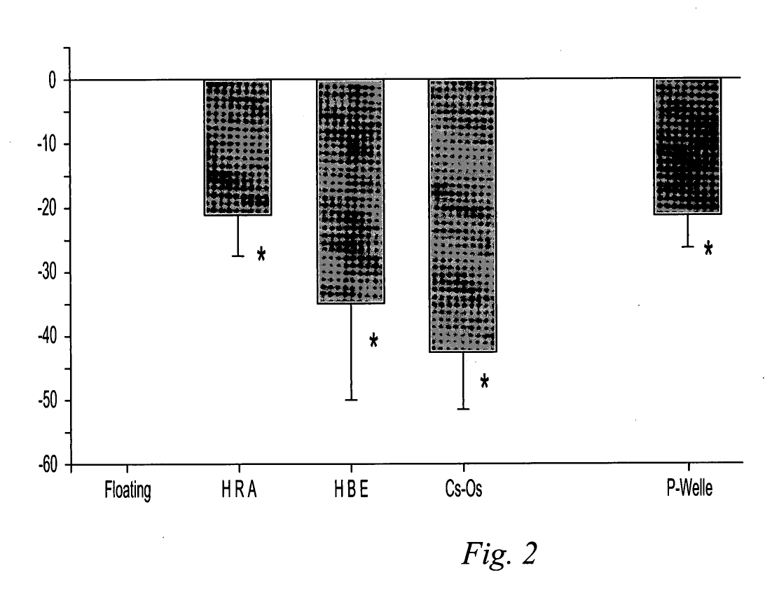

FIG. 2 shows the atrial intrinsic sensing commencement in the case of wall-located electrode positioning in the high right atrium (HRA), at the His's bundle (HBE) and at the ostium of the coronary sinus (Cs—Os; corresponds to the lower right atrium=URA) and in the surfaces—ECG der. 1 (P-wave) with respect to the sensing commencement by way of floating electrodes (Floating). This involves experimental data from 15 Merino sheep. Values identified by * are significantly later with respect to the sensing commencement by way of the floating electrodes.

The theory hitherto of “floating sensing” goes back to the investigations by Antonioli and Scalise. In accordance therewith the myocardial depolarization front is responsible, at the level of the floating electrodes, for the occurrence of the sensing signal. In accordance with that hitherto accepted theory floating sensing is a “local perception phenomenon”.

The results presented here, with simultaneous intrinsic signal perception by way of wall-located and floating electrodes cannot be explained with that theory. If floating sensing were only to reflect local activation at the level of the electrodes, atrial signal perception by way of wall-located electrodes in the HRA would have commence earlier. The intrinsic atrial excitation front begins in the sinus node and passes with a longitudinal propagation speed of 0.6 m/s by way of the atrium myocardium.

In dependence on the conduction time and the atrium size the sensing commencement differ in the present study in the HRA and the URA (Cs—Os) on average by 23 ms. As however perception by way of the ring electrodes floating in the right atrium in the central position (middle right atrium=MRA) begins on average 22 ms earlier than in the HRA, that cannot be the perception of the local myocardial depolarization front at the electrode level.

Signal perception which is 22 ms earlier approximately corresponds to a myocardial atrial excitation propagation distance which extends from the HRA to the Cs—Os. That earlier signal perception by way of floating electrodes therefore signifies that “floating sensing” involves farfield sensing, which occurs through the blood, in respect of activation, and not local myocardial activation at the electrode level.

That view is also supported by the observed decrease in the perceived amplitude heights from the HRA-position to the URA-position. That decrease in amplitude is the expression of the increase in distance from the sinus node. The results therefore contradict the previous view about a sole “local perception phenomenon” in relation to “floating sensing”.

Indications in respect of that view about “atrial floating sensing” are already to be found in the results from Antonioli, which however were not correctly interpreted. There, in regard to signal recording during the various atrial electrode spacings and positions, the amplitude height of the perceived ventricular signal are also specified. In that respect, an increase in the ventricular signal from 0.15 mV at the HRA-position to 0.46 mV at the URA-position was observed. That increasing ventricular signal was admittedly described by Antonioli as a “farfield signal”, but the changing atrial floating signal was described as “local perception” at the electrode level.

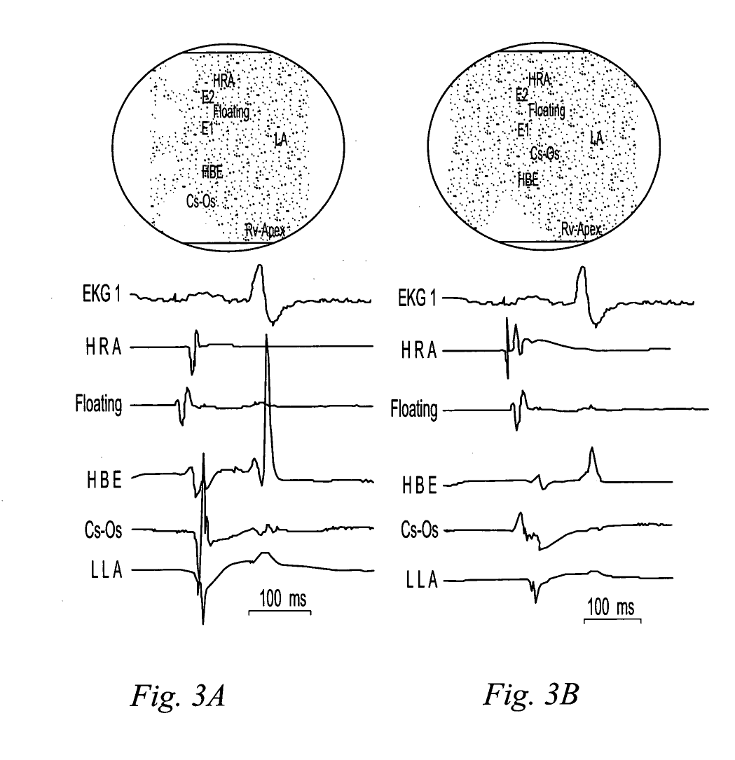

The results presented here relating to earlier signal perception by way of floating electrodes in comparison with all wall-located electrode positions contradict that theory from Antonioli. Results of investigations were carried out by the applicant show further proof in respect of that “farfield theory”. In such investigations, simultaneous electrogram recordings were implemented, more specifically both during a wall-located electrode position in the HRA and at the Cs—Os, and also after moving those electrodes away from the atrial wall so that they floated freely in the atrium. FIG. 3 shows in the upper part thereof an example of two RAO 30°-transillumination recordings. Recording A shows a wall-located position in the HRA and at the Cs—Os and a floating position in the middle atrium. Recording B shows a floating position in the HRA and above the Cs—Os and a floating position in the middle atrium.

With the floating positioning of the electrodes in the various stages of the right atrium the previously documented difference in the beginning of atrial signal perception disappears. The associated simultaneous recording of the intracardial derivations now causes the commencement of the atrial signals to appear almost at the same time.

The lower part of FIG. 3 shows, recorded from left to right, the simultaneous recording of the electrograms and the surfaces—ECG der. 1 in the case of wall-located positioning in the HRA and at the Cs—Os (A) and with a floating electrode position in the HRA and above the Cs—Os (B). The recorded signals in the HRA, at the Cs—Os and the floating ring electrodes are identified in red or marked by a boundary edge. The perpendicular line in each case identifies the commencement of signal perception by way of the floating electrodes E₁ and E₂. During the floating position the differing commencement of the atrial signal, during the wall-located position, is almost nullified.

Purely by way of example, in the illustrated embodiment there is a single wall-located electrode and two floating electrodes. It is however also possible to use a number differing therefrom of the respective type of electrode in order for example to be able better to determine the propagation behavior of the atrial signals.

The variation in the electrode position from wall-located (A) to floating (B) results in the loss of the perception which is different in respect of time. It can therefore be provided that the atrial signals are perceived not only by means of floating electrodes but also by means of a combination of floatingly and wall-locatedly arranged electrodes, in order in that way to be able to more accurately determine the propagation characteristics of the signals.

Based on this novel theory of “floating sensing” therefore it is also possible to perceive atrial ectopias at an earlier time than by way of wall-located electrodes. Earlier perception of signals permits an earlier reaction by stimulation and thereby possibly makes it possible to prevent the occurrence of auricular fibrillation or atrial tachycardia and ectopias.

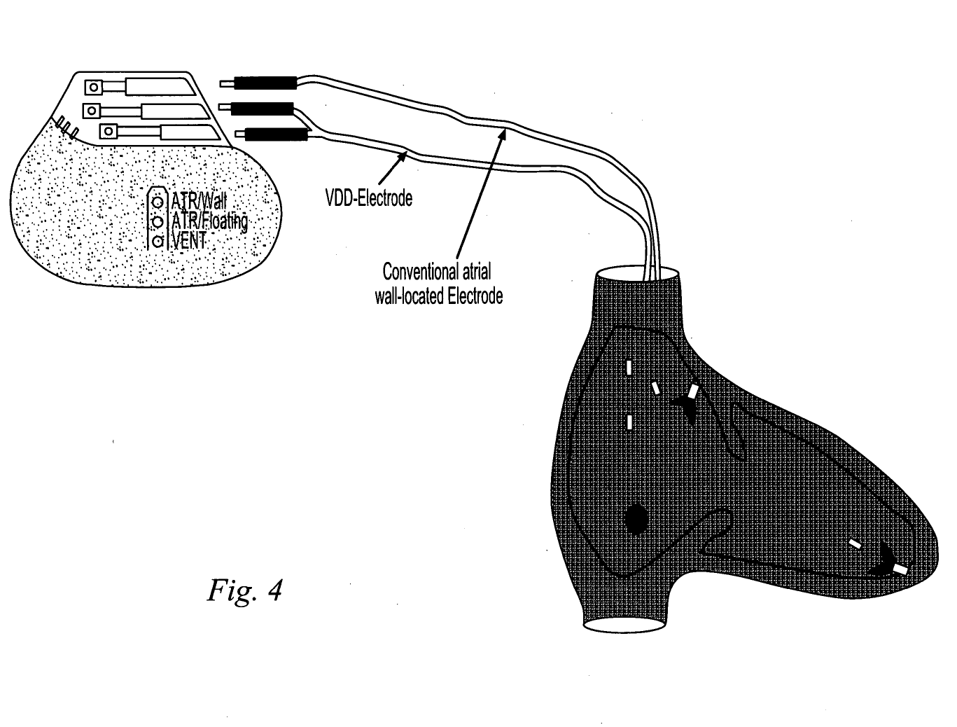

In accordance with the present proposal, based on those considerations, the options of floating sensing and wall-located stimulation are combined in a novel pacemaker arrangement. FIG. 4 shows the principle of the proposed AV-sequential cardiac pacemaker with the SPT-switch mode for optimization of early atrial signal perception (floating atrial ring electrodes), prevention by conventional stimulation (wall-located atrial electrode) and termination of atrial tachycardias or auricular fibrillation by temporary high-frequency floating stimulation (floating atrial ring electrodes).

The combination of a VDD-electrode with an additional atrial wall-located electrode affords the following possible options:

1. early perception of atrial signals by way of the floating electrodes,

2. additional possibility of differentiating the origin of the atrial signals by comparison of the simultaneous perceptions by way of the floating and wall-located electrodes, and

3. permitting earlier atrial stimulation after earlier perception both by way of the atrial wall-located electrode and also by way of the floating electrodes.

Mode 2: Pacing-Termination Mode

It is known from animal-experiment investigations by Allessie that even during auricular fibrillation local capture by high-frequency stimulation is possible. That principle of fast or high-frequency atrial stimulation for the termination of auricular fibrillation has already been integrated in a pacemaker system by way of wall-located electrodes. In that situation however the high-frequency stimulation by way of atrial wall-located electrodes did not lead to the hoped-for termination of auricular fibrillation, as Israel et al. found in a first study report about this novel pacemaker. The cause of this is that, in wall-located stimulation, even upon the attainment of local capture by the high-frequency stimulation, activation is limited to a maximum area of a diameter of 5 cm, as Allessie was already able to show. However that simultaneously activated area is generally not sufficient to terminate auricular fibrillation which has occurred. The area of simultaneous activation which results in interruption in auricular fibrillation must be significantly greater.

In animal-experiment studies the applicant was able to establish that atrial floating stimulation, irrespective of the stimulation concept (OLBI=overlapping biphasic impulse, BIMOS=bidirectional monophasic impulse, conventionally bipolar or unipolar with markedly higher output), results in large-area simultaneous activation of the atrial myocardium.

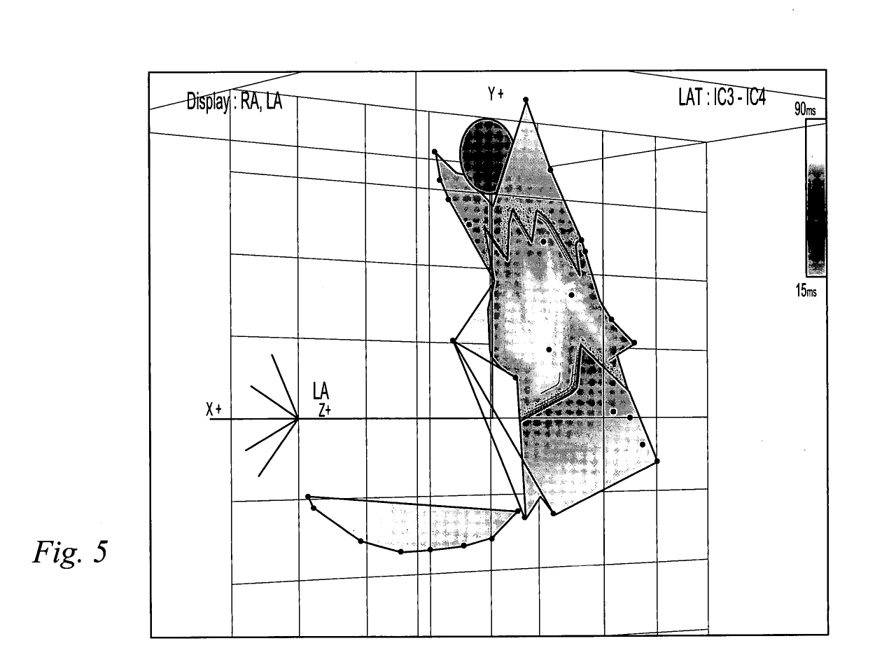

By means of a novel mapping system (CARTO system), the applicant was able to represent the atrial simultaneous activation area under floating stimulation: FIG. 5 shows as an example of an illustration of the activation sequences during floating atrial stimulation which results in large-area simultaneous activation of the atrial myocardium, a posterior view of a CARTO mapping recording with atrial floating stimulation. The region of the earliest activation is illustrated in red or by hatching while the blue or square-marked area identifies the region of latest activation. The procedure virtually involves a belt-shaped simultaneous early activation of the entire right atrium including the interatrial septum. The simultaneously activated area is consequently a multiple larger, in comparison with wall-located stimulation.

It was further possible to establish in a large number of studies that the OLBI principle developed by the applicant, in comparison with a conventional unipolar or bipolar stimulation configuration, affords stable atrial floating stimulation, with practically acceptable stimulus thresholds.

The principle of floating stimulation by means of OLBI stimulation has hitherto not yet gained general acceptance as, in spite of the significant stimulus threshold reduction in comparison with stimulation with conventional impulse configurations, in about 25% of cases, intermittent diaphragmal co-stimulation has also occurred. In accordance with the present proposal therefore stimulation is basically effected, as mentioned hereinbefore, by means of the wall-located electrode in a per se known and as pain-free form as possible, for the patient.

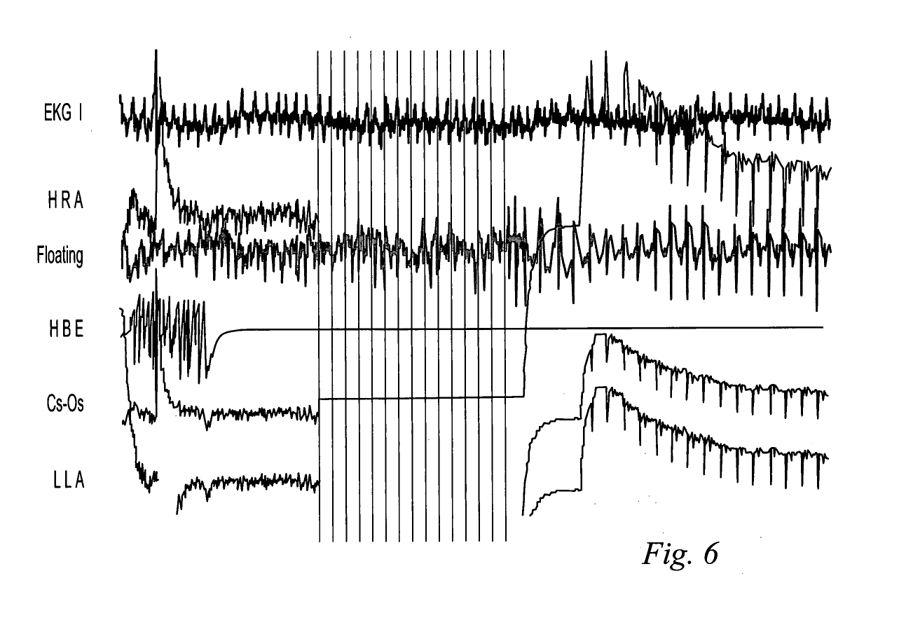

In new, hitherto unpublished animal-experiment investigations however the applicant was able to establish that is possible to terminate auricular fibrillation with the large-area atrial high-frequency floating stimulation. FIG. 6 shows an example of surface ECG and intracardial recordings of a termination of auricular fibrillation by using the large-area floating atrial stimulation at high frequency, on the basis of an example of an animal-experiment simultaneous recording, which is registered from left to right, with a wall-located electrode position in the high right atrium (HRA), at the His's bundle (HBE), at the ostium of the coronary sinus (Cs—Os; this corresponds to the lower right atrium—URA) and at the wall of the left atrium (LLA) and a floating electrode position in the middle right atrium (floating) and in the surfaces—ECG der. 1 (P-wave) during induced auricular fibrillation. In the middle portion high-frequency stimulation is effected by way of the floating electrodes with the OLBI-configuration, thereby affording termination of the auricular fibrillation, as is apparent from the rear portion of the recordings.

Therefore, for those respectively time-limited situations of use for the termination of paroxysmally occurring complaints such as auricular fibrillation or cardial tachycardias, the pacemaker arrangement can be switched over to the second mode in which stimulation is effected solely by way of the floating electrodes or by way of a combination of floating and wall-located electrodes, in which case diaphragmal co-stimulation which possibly occurs for that time-limited situation of use can be readily tolerated, in consideration of the advantages which can be achieved.

Based on those observations the novelty of the present proposal is that the principle of floating stimulation, irrespective of the mode involved (OLBI, BIMOS or conventional), is applied to high-frequency temporary stimulation for the termination of auricular fibrillation and atrial tachycardias. In contrast to stimulation by way of exclusively wall-located electrodes, in the detection of auricular fibrillation or atrial tachycardias, stimulation is simultaneously effected over a large area.

By virtue of the combination of the electrode arrangement of floating and wall-located electrodes on the one hand the advantages of floating sensing (earlier signal perception than with wall-located electrodes) and the advantages of tried-and-tested wall-located atrial stimulation (no diaphragmal co-stimulation) in the absence of atrial tachycardias are linked to the advantages of large-area high-frequency stimulation for terminating auricular fibrillation and atrial tachycardias.

The electrodes that can be used are unipolar and/or bipolar electrodes so that in the present text in part the term “electrode” and in part the term “electrodes” are used, without in that respect in each case meaning exclusively the use of only one or only two or more electrodes. To sum up, attention is directed to the following, regarding the principle of the present proposal of “S-P-T-switch mode”:

The principle of the cardiac pacemaker with an SPT-switch mode is that, contrary to previous pacemaker systems, the proposed system represents a combination of a VDD pacemaker system and an additional conventional wall-located atrial electrode (FIG. 4). In that respect atrial signal perception is always effected by way of the floating ring electrodes of the VDD-electrode and possibly simultaneously by way of the wall-located electrode. Depending on the respective placement of the wall-located electrode information about the location of origin of the atrial signal (sinus rhythm, right-atrial or left-atrial ectopia, etc.) can be furnished from the time difference between signal perception by way of the floating electrodes and signal perception by way of the wall-located electrodes.

Normal atrial stimulation is effected by way of the wall-located electrode in conventional manner. If atrial tachycardias or auricular fibrillation are perceived the arrangement switches over to the termination mode. That evaluation is effected for example on the basis of the frequency of the perceived signals. In that respect it is possible to provide for individual adaptation of the frequency limit value to the individual patients: frequencies above for example about 150 Hz or 180 Hz can be assessed as an indication of atrial tachycardias or auricular fibrillation. Optionally, in place of—or combined with—a frequency limit value which is fixedly predetermined or set on an individual patient basis, another “trigger” can cause the pacemaker to switch over to the termination mode: that switching-over action can take place for example in dependence on the origin or the propagation characteristics of the atrial signals, in which respect such perception is possible by comparison of the simultaneous perceptions by way of the floating and the wall-located electrodes.

In the termination mode it is possible to apply either given stimulation algorithms or also impulse series of different high frequencies, of various durations. Purely by way of example, in which respect also other cycle lengths may be advantageous, stimulations with a cycle time of between 30 and 100 ms can be considered as high-frequency impulse series, in contrast to low-frequency stimulations with a cycle time of about 600 ms.

In the termination mode the impulses can be applied either between the wall-located and the floating electrode or electrodes or only between the floating electrodes. The impulses which are used in that situation can represent both conventional impulse configurations and also special impulse configurations such as OLBI or BIMOS. However other forms of impulse application either by way of the floating ring electrodes only or by way of the wall-located and floating electrodes jointly can also be envisaged.

1. A cardiac pacemaker arrangement comprising.
an electrode arranged floatingly in the atrium;
a circuit for perceiving atrial signals; and
a circuit for stimulating the atrial myocardium by means of the electrode,
characterized in that in addition there is provided a wall-located electrode, and stimulation is effected by means of the wall-located electrode if the circuit, upon perceiving atrial signals, does not detect high-frequency irregularities
such as auricular fibrillation or atrial tachycardias
as on the basis of inadmissibly high signal frequencies,
and stimulation is effected by means of the floating electrode if the circuit, upon perceiving atrial signals, detects said high-frequency irregularities.

2. A pacemaker arrangement as set forth in claim 1 characterized in that stimulation is effected by means of the floating electrode at high frequency, such as with a cycle length of between about 30 and 100 ms.

3. A pacemaker arrangement as set forth in claim 1 characterized in that there are provided two or more floating electrodes.

4. A pacemaker arrangement as set forth in claim 1 characterized in that there is provided a single wall-located electrode.

5. A pacemaker arrangement as set forth in claim 1 characterized in that switching over to stimulation by means of the floating electrode is effected upon a perception of atrial tachycardias or higher-frequency signals.

6. A pacemaker arrangement as set forth in claim 1 characterized in that the floating electrode is associated as a sensor with the circuit for perceiving atrial signals.

7. A pacemaker arrangement as set forth in claim 1 characterized in that the wall-located electrode is associated as a sensor with the circuit for perceiving atrial signals.

8. A method of controlling a cardiac pacemaker wherein atrial signals are perceived by means of an electrode arranged in the atrium of the heart and are evaluated in a circuit of the cardiac pacemaker, and wherein in dependence on the perceived signals the circuit triggers stimulation of the myocardium by means of an electrode arranged in the atrium of the heart,
characterized in that the atrial signals are perceived by means of a floating electrode,
stimulation of the myocardium is basically effected by means of a wall-located electrode, and
if the atrial signals are evaluated by the circuit as tachycardias or auricular fibrillation stimulation of the myocardium is effected by means of a floating electrode.

9. A method as set forth in claim 8 characterized in that the circuit evaluates atrial signals as tachycardias or auricular fibrillation if the signal frequency is about 150 Hz or higher.

10. A method as set forth in claim 8 characterized in that stimulation is effected by means of the floating electrode at a high frequency such as with a cycle length of between about 30 and 100 ms.

11. A pacemaker arrangement as set forth in claim 2 characterized in that there are provided two or more floating electrodes.

12. A pacemaker arrangement as set forth in claim 2 characterized in that there is provided a single wall-located electrode.

13. A pacemaker arrangement as set forth in claim 3 characterized in that there is provided a single wall-located electrode.

14. A pacemaker arrangement as set forth in claim 2 characterized in that switching over to stimulation by means of the floating electrode is effected upon a perception of atrial tachycardias or higher-frequency signals.

15. A pacemaker arrangement as set forth in claim 3 characterized in that switching over to stimulation by means of the floating electrode is effected upon a perception of atrial tachycardias or higher-frequency signals.

16. A pacemaker arrangement as set forth in claim 4 characterized in that switching over to stimulation by means of the floating electrode is effected upon a perception of atrial tachycardias or higher-frequency signals.

17. A pacemaker arrangement as set forth in claim 2 characterized in that the floating electrode is associated as a sensor with the circuit for perceiving atrial signals.

18. A pacemaker arrangement as set forth in claim 3 characterized in that the floating electrode is associated as a sensor with the circuit for perceiving atrial signals.

19. A pacemaker arrangement as set forth in claim 4 characterized in that the floating electrode is associated as a sensor with the circuit for perceiving atrial signals.

20. A pacemaker arrangement as set forth in claim 5 characterized in that the floating electrode is associated as a sensor with the circuit for perceiving atrial signals.

21. A pacemaker arrangement as set forth in claim 2 characterized in that the wall-located electrode is associated as a sensor with the circuit for perceiving atrial signals.

22. A pacemaker arrangement as set forth in claim 3 characterized in that the wall-located electrode is associated as a sensor with the circuit for perceiving atrial signals.

23. A pacemaker arrangement as set forth in claim 4 characterized in that the wall-located electrode is associated as a sensor with the circuit for perceiving atrial signals.

24. A pacemaker arrangement as set forth in claim 5 characterized in that the wall-located electrode is associated as a sensor with the circuit for perceiving atrial signals.

25. A pacemaker arrangement as set forth in claim 6 characterized in that the wall-located electrode is associated as a sensor with the circuit for perceiving atrial signals.

26. A method as set forth in claim 9 characterized in that stimulation is effected by means of the floating electrode at a high frequency such as with a cycle length of between about 30 and 100 ms.
